(19) United States
(12) Patent Application Publication
(10) Pub. No.: US 2026/0041581 A1
(43) Pub. Date: Feb. 12, 2026

(54) Contrast Therapy Machine

(71) Applicant: Joseph Bommarito, Petosky, MI (US)

(72) Inventor: Joseph Bommarito, Petosky, MI (US)

(21) Appl. No.: 19/295,569

(22) Filed: Aug. 9, 2025

Related U.S. Application Data
(60) Provisional application No. 63/681,545, filed on Aug. 9, 2024.

(51) Int. Cl. A61F 7/00; A61L 2/02; A61L 2/07; A61L 2/10; A61L 2/20

(52) U.S. Cl. CPC A61F 7/0053; A61L 2/022; A61L 2/07; A61L 2/10; A61L 2/202; A61F 2007/0093; A61F 2007/0096; A61L 2202/14; A61L 2202/20

(57) ABSTRACT

A contrast therapy machine is disclosed. The contrast therapy machine includes a cavity that is sized and shaped to accommodate one or more human bodies. Single user contrast therapy machines may include an opening through a top surface permitting the human body from the neck up to be outside of the contrast therapy machine while it is use. The contrast therapy machine includes a heater and a cooler. The contrast therapy machine can switch automatically or be manually switched between one or more cooling cycles and one or more heating cycles. Heating cycles and cooling cycles may be customized.

### RELATED CASES

This application claims the benefit of U.S. Provisional Patent Application No. 63/681,545, filed on Aug. 9, 2024, which is hereby incorporated by reference in its entirety.

### TECHNICAL FIELD

The present disclosure relates to a contrast therapy machine that includes a cavity sized and shaped to accommodate at least one body, such as a human body, or animal body, such as a horse. The contrast therapy machine causes heating cycles and cooling cycles to be manually or automatically applied to person(s) in the cavity of the contrast therapy machine. The contrast therapy machine may be programmed to switch back and forth between a selected number of heating cycles at selected temperatures and a selected number of cooling cycles at selected temperatures.

### BACKGROUND

Contrast therapy generally involves the therapeutic application of heat and cold to the human body in alternating cycles. It is generally understood that the application of heat can safely start recovery of the human body and improve circulation. It is also generally understood that application of cold can safely start recovery of the human body and reduce inflammation and improve fat conversion.

The temperatures and the durations selected for a given heating or cooling cycle may depend on a particular patient's healing needs as directed by a medical professional and/or tolerance level. The number of heating or cooling cycles may similarly depend. Generally, it is understood that time between peak temperatures of a heating cycle and nadir temperatures of a cooling cycle should be long enough to avoid shock but short enough to optimize for efficacy.

Many different approaches have been used for the healing effects of the application of heat and the application of cold to the human body for healing. For heat, these include hot compresses, heat lamps, saunas, whirlpools, and other known techniques and/or apparatuses. For cold, they include ice, cold plunges, cryotherapy, and other known techniques and/or apparatuses. Many of these known techniques and/or apparatus involve challenges.

For heat, hot compresses and heat lamps can cause burns, blistering, tissue damage, and other issues. Saunas may be set at a standard temperature that can be too hot for some people and too cold for others. In some cases, a sauna that is too hot for a particular person can present a health risk. Additionally, many traditional saunas can take a substantial amount of time to reach a peak desired temperature. In some instances, that time can be 15 to 20 minutes or longer.

For cold, cold plunges can shock the body as the body rapidly drops core temperature. This may increase the risks of cardiac arrest, drowning, tissue damage, and hypothermia. Similarly, the direct application of ice can cause frostbite and/or damage tissue. Cryotherapy, with cooling mechanisms such as the use of liquid nitrogen or electrical components such as, for example, heat sink coolers, heat pipe coolers, vapor compression coolers, thermoelectric coolers (TEC), at temperatures as low as −320 Fahrenheit may increase the risks of frostbite, frozen limbs, tissue and cell damage, hypothermia, as well as other conditions.

While any of these existing techniques for the application of heat and cold may work for a given situation, these existing techniques by themselves are not known to provide contrast therapy. Furthermore, using existing techniques, a person seeking contrast therapy may need to be well enough to move between a heating apparatus and a cooling apparatus quickly. Many people needing contrast therapy are not in the physical condition to do that, because many have an illness or one or more injuries to one or more parts of their bodies. Additionally, many existing techniques that can be used with contrast therapy are not programmable, such as the manual application of hot compresses, heat lamps, cold plunge, ice and other heating or cooling apparatuses.

Additionally, some traditional contrast therapy machines require submersion into liquid water. Such water submersion machines may be expensive to operate, slow to make desired temperature adjustments, limited in temperature peak and nadir ranges, and/or difficult to clean or sterilize.

There exists a need for a contrast therapy machine that addresses at least one of the challenges presented by current systems and methods.

### BRIEF DESCRIPTION OF DRAWINGS

FIG. 1 is a perspective view of an exterior of an exemplary contrast therapy machine in a closed position.

FIG. 2 is a perspective view of an interior of an exemplary contrast therapy machine in an open position.

FIG. 3 is a perspective view of an exemplary contrast therapy machine in use.

FIG. 4 is a cut away view of a wall section in an exemplary contrast therapy machine.

FIG. 5 is a schematic of a contrast therapy system.

FIG. 6 is a control box for an exemplary contrast therapy machine.

FIG. 7 is an exemplary heater.

FIG. 8 is an exemplary booster heater.

FIG. 9 is an exemplary cooler.

FIG. 10 is an exemplary seat base and seat.

FIG. 11 is an exemplary seat base and seat.

### DETAILED DESCRIPTION

As described herein, contrast therapy may be delivered to a body, such as a human body as depicted, but it could also include an animal body, such as a horse, that is at least partially within a contrast therapy machine. Contrast therapy as used herein involves one or more heating cycles and one or more cooling cycles. The contrast therapy may be customized depending on a person's therapy needs and tolerances. That is, users may create a tailored contrast therapy program session by manually or remotely inputting preferences for particular parameters.

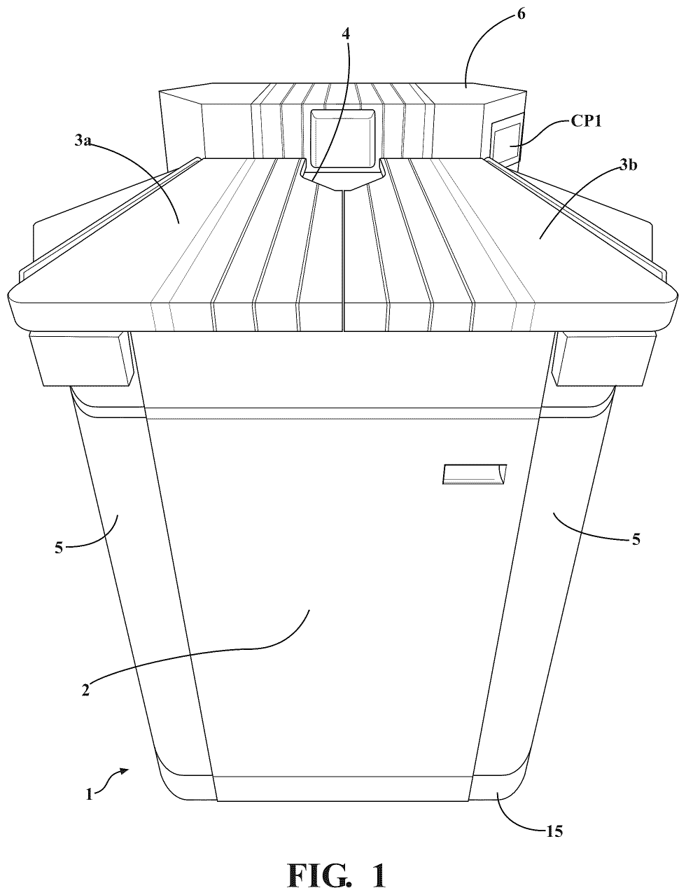

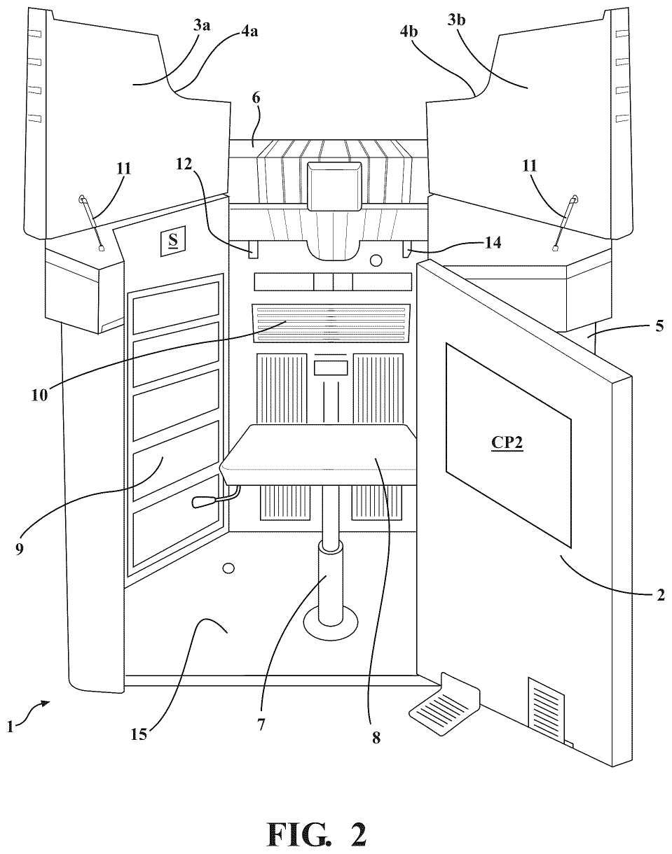

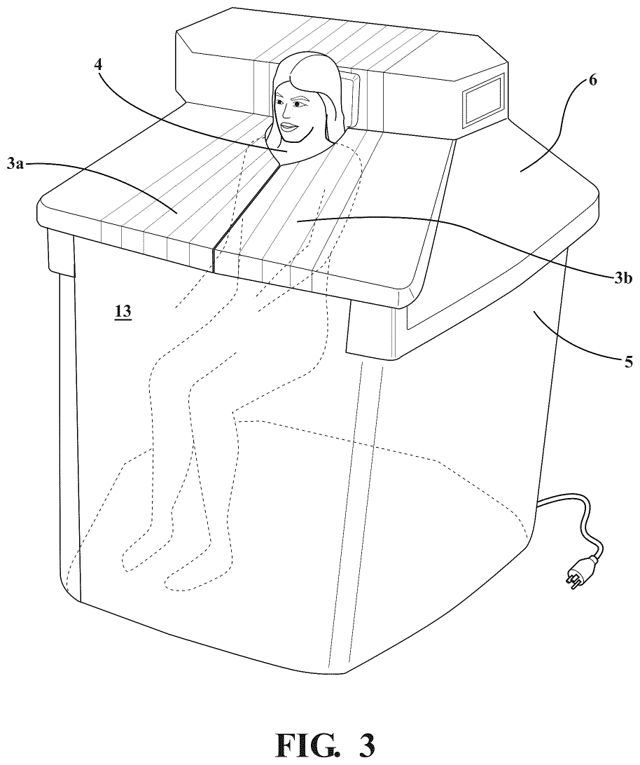

Turning now to the drawings for a general overview, an exemplary non-limiting embodiment of a contrast therapy machine 1 is shown in FIGS. 1-3. In FIG. 1, the exemplary contrast therapy machine 1 is depicted in a fully closed position. In FIG. 2, the exemplary contrast therapy machine 1 is depicted in a fully open position. In FIG. 3, the exemplary contrast therapy machine 1 is depicted in use in a fully closed position.

Turning back to FIG. 1, one or more walls 5 extend from a base 15. A door 2 is attached with or supported by one or more walls 5. The walls 5 and the closed door 2 define a cavity therebetween, sized and shaped to accommodate a single human body. Embodiments are contemplated to accommodate a plurality of human bodies. The walls 5 may be straight or curved. The walls 5, door 2, and base 15 may be constructed of the same or different materials, which may include one or more of wood, metal, and plastic. The walls 5, door 2, and base 15 may be solid or hollow, insulated in whole or in part, integral or modular, and they may be formed, built, and constructed in whole or in part using manufacturing techniques like 3D printing, extrusion, injection molding, and other techniques. The walls 5, door 2, and base 15 may be joined, attached, or connected using any of a number of known mechanical connection devices and connection techniques like screws, nails, bolts, and also by glues, and combinations of mechanical attachments and adhesives.

In some embodiments, a control panel CP1 is in or on or otherwise supported by wall 5 or top surface 6 for a user to manually enter input to create a desired contrast therapy program session. Inputs for many parameters are contemplated. By way of non-limiting example, user may customize parameters such as number of heating cycles, duration of heating cycles, temperature peaks for heating cycles, number of cooling cycles, duration of cooling cycles, temperature nadirs for cooling cycles, and types of alerts requested for selected events. Parameters might also permit launching of a sanitizing cycle before or after use, as well as selection of duration of a sanitizing cycle. These parameters are described in more detail elsewhere in this disclosure.

Exemplary alerts for selected events may be auditory, including voice or tones, haptic, including vibration of one or more element in the contrast therapy machine 1, or visual, including light or flashing light indicators. Alerts may be transmitted wirelessly, such as text messages to a provided mobile number. Events where alerts may be requested may be a when a contrast therapy program session begins, a beginning or end of a heating cycle, a beginning or end of a cooling cycle, reaching of a peak temperature, reaching of a nadir temperature, and/or preliminary warning signals in advance of any of the foregoing. The control panel CP1 is in electrical communication with a controller which receives input (whether manually or wirelessly) and processes the input to generate one or more signals or control sequences to cause a contrast therapy program session to run according to the input. Sensed temperature is sent to and processed by the controller to compare the sensed temperature data to input. In some cases, the comparison will cause a determination if a heater or cooler must go on or off to for a temperature to be maintained with a tolerance range of input temperature. The processor will generate output thereby directly or indirectly causing action according to occur. are we missing a work in this sentence?

Sensed temperature data may include instantaneous or real-time temperature at a specific location within the cavity, time-averaged temperature data calculated over a moving interval, the rate of temperature change over time (i.e., the derivative of temperature with respect to time), and whether a preset temperature threshold—such as a peak heating temperature or nadir cooling temperature—has been reached or exceeded. In some cases, sensed temperature data may also include ambient air temperature, surface temperature, or radiative temperature. Output from the temperature sensor may be provided to the controller in either analog or digital form using known communication protocols for use in adjusting heaters and coolers to closely approach the desired parameters based on input for a given contrast therapy program session.

At least a portion of the top surface 6 covers the cavity and acts as a cover. “Top surface” is used for convenience and does not require the top surface 6 to be flat or made from the same material throughout or even share a common shape or surface. As depicted, at least a portion of the top surface 6 covering the cavity is openable and closable by doors 3a and 3b. In some embodiments, the top surface 6 is not openable or closeable. In the depicted embodiment, in the closed position, an opening 4 is positioned in the top surface and runs through the top surface entirely, such opening being sized and shaped to accommodate a human neck. In such embodiments, it is contemplated that that the opening 4 may optionally include a compliant barrier such as a rubber flap or soft brush bristle to help maintain a thermal barrier between an interior of the contrast therapy machine 1 and the atmosphere. Other such compliant barrier materials include LYCRA, spandex, nylon or other known natural and/or synthetic woven and nonwoven materials. It is not expected that the thermal barrier would be airtight. It is also contemplated that users may use a towel or other covering to support a thermal barrier at an outer circumference of the opening 4. It is contemplated that at least some embodiments will not have an opening 4, and that one or more persons would be fully enclosed in the cavity of the contrast therapy machine 1 when in use. FIG. 1 shows a fully closed position because doors 2, 3a, and 3b are all closed. A user can open one or both of doors 3a and 3b to then open door 2 to transition into a fully open position. In some embodiments, door 2 can be opened even when doors 3a and 3b, if present as part of top surface 6, are closed.

In FIG. 2, exemplary contrast therapy machine 1 is in a fully open position with the interior of the contrast therapy machine 1 exposed. That is, door 2 is open and doors 3a and 3b are open. An overview of the illustrated components follows, with the understanding that the illustrated components may be present or absent in alternative examples and in any number of combinations. Starting from the bottom of FIG. 2 and moving generally clockwise, base 15 provides support for walls 5, which in turn support door 2. Base 15 also supports seat base 7 and seat 8, directly or indirectly. Base 15 may optionally include one or more bores for airflow to permit venting or introduction of heated air, or venting or introduction of cooled air. Walls 5 may support one or more heaters 9 that form part of a heating system. Walls 5 may further support one or more sensors S for detecting temperature data within the cavity while the contrast therapy machine 1 is in use. Walls 5 may further support one more coolers 10 that form part of a cooling system. Shock-style door hinges 11 are shown supporting doors 3a and 3b in an open position. Continuing clockwise movement, booster heater 12 may optionally form part of the heating system to accelerate raising of temperatures during a heating cycle. Next, door 3a includes contouring 4a to form opening 4 (FIGS. 1 and 3) for a neck when closed and in combination with contouring 4b on door 3b. Next, a sanitizer 14 is supported by wall 5.

Now, more details of components in FIG. 2 are provided. Door 2 is shown as a single door with its hinges not being visible. Many known hinges and optionally locks and locking mechanisms are contemplated for use with door 2. Door 2 may have attachments on its interior surface and/or on exterior surface for opening the door 2. Although not shown in FIG. 2, standard handles and recess-based handles are contemplated for use with door 2. Although door 2 is shown as being supported from an edge, other configurations are contemplated. For example, saloon style swinging doors may be used in some embodiments.

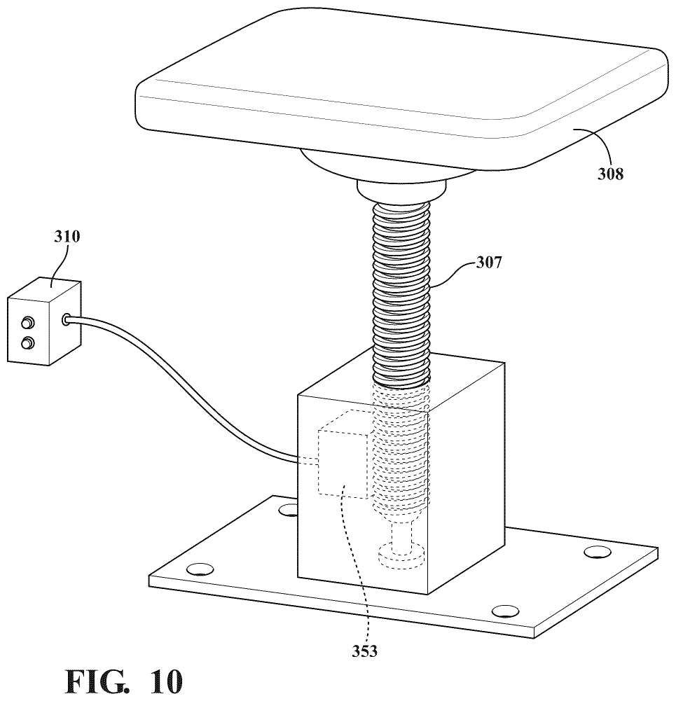

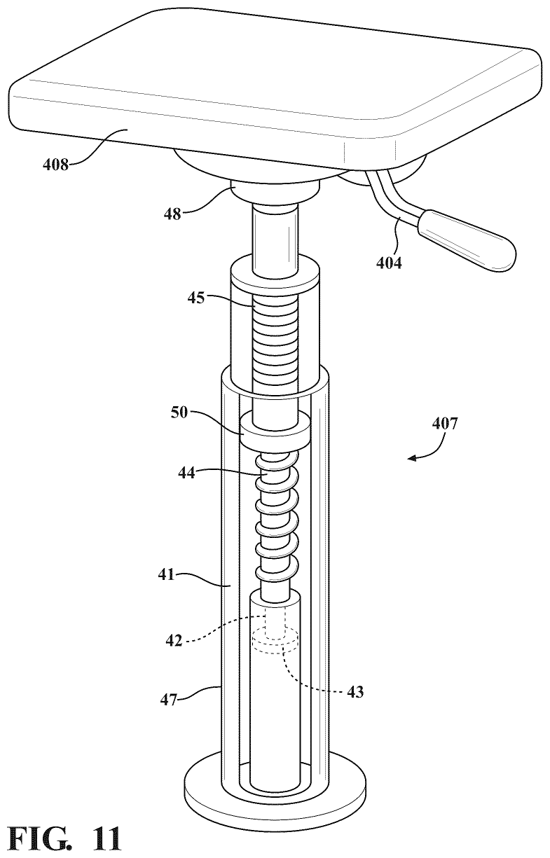

Seat base 7 and seat 8, as depicted, form a vertically adjustable single-person seating system. Seat base 7 may have its own seat controller for electronic vertical adjustment. It is contemplated that seat base 7 and/or seat 8 may be manually or electronically adjustable other than vertically (swivel, tilt, etc.), or that seat base 7 and seat 8 may be stationary and not adjustable. Such stationary bases 7 and seats 8 may be used, for example, in embodiments where the contrast therapy machine 1 does not have doors 3a or 3b nor an opening 4 for a neck. Seat 8 can be backless as illustrated to allow unobstructed access to a person's back for heating and cooling. Optionally, seats 8 may provide physical back support and include optional adjustable lumbar support. Seats 8 may be shaped as benches in smaller contrast therapy machines 1, permitting a person to lay down within a cavity. In larger contrast therapy machines 1, seats 8 may be shaped as benches to accommodate more than one person in a seated position. Seats 8 may be hard-surfaced or they may be cushioned and/or covered with a fabric or material. In some embodiments, the fabric or material may include plastic or faux leather or other material that is easy to clean and/or disinfect. Suitable examples for seat base 7 and seat 8 are included and described in FIGS. 10 and 11.

There may be one or more heater 9 supported directly or indirectly by wall 5. As shown, heaters 9 are infrared heating panels, but other heaters may be suitable. Other heaters 9 may include an electric sauna heater (resistive element+stones), a gas-powered sauna heater (natural gas or propane), a ceramic infrared heaters (alternative IR tech), or other known heating mechanisms, or combinations of one more of those devices. Heaters 9 may comprise some or all of a heating system, which may also include one or more booster heater, one or more fans, and in some cases, one or more ducts or channels to blow heated air into the cavity. Optionally, where one or more channels or ducts lead into the cavity, a nozzle or other duct/channel covering may be adjustably or fixedly targeted toward the areas where part or all of a human body would be expected to sit or lay down within the enclosure. Heaters 9 may be any of a number of heating devices suitable for use in contrast therapy. Heaters 9 may be in electrical communication with the controller and can be actuated, operated, and deactivated according to input and/or sensed temperature data processed by the controller.

There may be one or more cooler 10 may be supported directly or indirectly by wall 5. As shown, cooler 10 may be all of or part of a cooling system. The cooling system may allow venting of heated air and may include at least one fan. In some embodiments, the cooling system may be fitted with ducts or channels through which cooled air can be moved and directed to the cavity. Cooler 10 may be any of a number of cooling devices suitable for use in contrast therapy. In some embodiments, cooler 10 is an air conditioning unit such as a commercially available indoor/outdoor air conditioning window unit. Other suitable coolers may include a mini split air conditioning unit, Peltier thermoelectric cooler, a vapor compression refrigeration unit, an evaporative cooler, a cold air blower/cryo fan, or cold plate or cold wall panels, or other known cooling mechanisms, or combinations of one or more of those devices. Cooler 10 may be in electrical communication with the controller and can be actuated, operated, and deactivated according to input and/or sensed temperature data processed by the controller.

One or more temperature sensors S may optionally be installed and positioned to sense temperature data within the cavity. Sensors S may be one or more of a thermostat, thermistor, or digital temperature probe, or other temperature sensor in electrical communication with the controller. Sensors S may be configured to send temperature data to the controller in real time when sensed (whether via wire or wirelessly), which controller is configured to process the sensed temperature data. Such sensed temperature data may provide a feedback loop in this manner. Output from the controller, as modified by input from sensed temperature data, causes directly or indirectly heaters and coolers within the heating system and cooling system to adjust to reach and/or maintain the selected peak and nadir temperatures for the selected duration. That is, for example, periodically in a 15 minute heating cycle, certain heaters 9 may get turned down or turned off (and turned back on again) to maintain the selected peak temperature for a select duration. To this end, it is expected that there may be some tolerances and allowances for not maintaining a specific temperature precisely, especially in embodiments where there is a neck opening and the cavity is not airtight. Such tolerances may be +/−5 degrees F., or +/−3 degrees F., for example. Additionally, if a selected input peak or nadir temperature is outside the range of what the heating system or cooling system can physically reach, then the software may be configured to select the highest reachable temperature or the lowest reachable temperature and use those limits in place of selected temperature peaks and nadirs, including in instances where the difference is greater than the usual tolerance/allowance range. In some embodiments, user input will be restricted to machine limits or shy of such limits so that users cannot enter temperatures higher or lower than can be reached. Additionally, each contrast therapy machine 1 may optionally have a kill switch reachable from the seat 8. Optionally, an additional kill switch may be placed on an exterior of the contrast therapy machine 1 to be reached by third parties. All kill switches are in electrical communication with the controller and by extension the heating system and its components and the cooling system and its components.

Door hinges 11 as illustrated are shock-style hinges 11. These are sometimes referred to as damped hinges or soft-close hinges. They may be hydraulic or gas spring hinges, and may have integrated biasing or damping mechanisms to assist in the opening and closing of doors 3a and 3b. Other types of hinges are contemplated, whether or not shock style. Such hinges include but are not limited to butt hinges, plano hinges (continuous hinges), barrel hinges, pivot hinges, living hinges, spring hinges, double action hinges, concealed hinges sometimes referred to as a European hinge, butterfly hinges, rising butt hinges, self-closing hinges, and hinges with electrical feedthroughs to accept instructions sent by the controller to commence opening, closing, and optionally locking according to received input parameters, for example, when a contrast therapy program session is about to begin or has recently concluded.

Doors 3a and 3b have been described herein. The contouring 4a and 4b combine to create an opening 4 that is within and through a portion of top surface 6, and the combined contouring of 4a and 4b can take on any number of shapes and sizes, but may be shaped and sized to accommodate a range of human neck sizes and shapes. As described supra, the contouring 4a and 4b may optionally include a soft barrier to bridge the gap between smaller necks and edges of doors 3a and 3b to help the cavity maintain its desired thermal environment when in use.

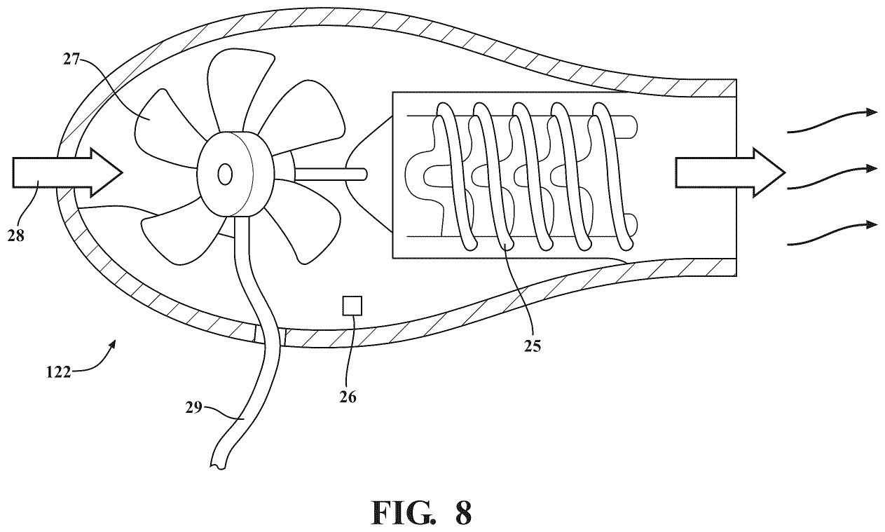

Booster heater 12, as depicted symbolically in FIG. 2, an example of which is described in FIG. 8, is an optional component of the heating system. Booster heater 12 may comprise a forced air heater, which may be used to increase the speed at which temperatures are raised after a heating cycle is initiated, whether from ambient temperatures or from the end of a cooling cycle. Additional types of heaters can be adapted to serve as booster heater 12 if suitable for use in a contrast therapy machine. Such heaters may include ceramic space heaters, Infrared heaters, PTC (Positive Temperature Coefficient) air heaters, hydronic booster heaters, duct or in-line booster heaters, electric baseboard boosters, radiant floor or additional wall panel heaters, or combinations of one or more such heaters. Booster heaters 12 may be in electrical communication with the controller and can be actuated, operated, and deactivated according to input and/or sensed temperature data processed by the controller.

Sanitizer 14 may be one or more of any number of sanitizing devices that can kill bacteria within the cavity of the contrast therapy machine 1. By way of non-limiting example, a sanitizer 14 can be one or more of a UV-C light sanitizer, an ozone generator, a dry steam cleaner, an electro static sprayer, a HEPA air purifier, or an activated carbon air purifier. Still other sanitizer 14 devices are contemplated, including a plasma ionizer, hydrogen peroxide foggers, and a photocatalytic oxidation (PCO) device. The sanitizer 14 may be in electrical communication with a controller that processes input to generate a signal to initiate sanitizing. Optional inputs may include duration of a sanitizing cycle before or after a contrast therapy program session and/or the type of sanitizing requested if more than one sanitizer 14 is installed to sanitize the cavity before or after usage.

Optional control panel CP2 may be included within the cavity. In embodiments where there is no opening 4, CP2 may allow real-time input and editing or cancelling of an initially executed contrast therapy program session. If editing, adjustments may be made to one or more of peak temperatures, nadir temperatures, durations, alerts, and other programmable parameters.

In FIG. 3, a person is illustrated using a single-person embodiment or non-limiting example of contrast therapy machine 1. Door 2 is closed, as are doors 3a and 3b. The person's neck extends through opening 4, and the portion of person's body beneath their neck is hidden with the cavity 13.

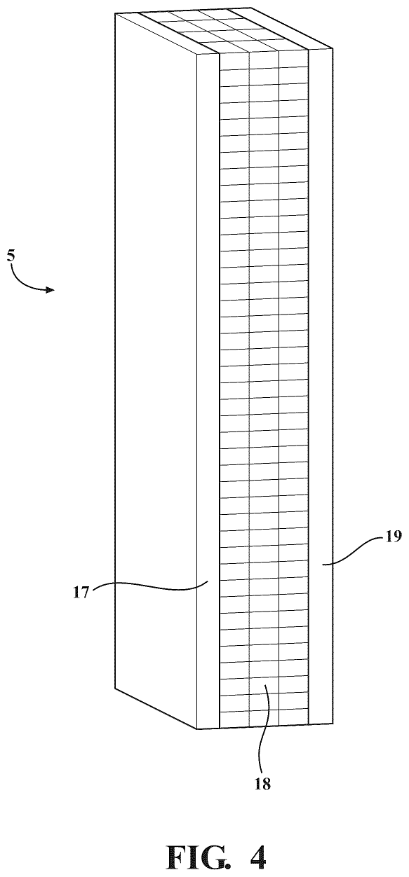

In FIG. 4, an exemplary cut-away of an insulated portion of wall 5 is shown. It is contemplated that wall 5 includes an inner wall 19 and an outer wall 17 with insulation 18 positioned therebetween. The outer wall 17 may be made of one or more of plastic, metal, wood, laminate or other material that include a commercially acceptable aesthetic and is readily cleanable. The inner walls 19 may be constructed of one or more suitable materials, such as, for example, of wood, metal, plastic, ceramic or fiberglass and/or a combination of one or more of those materials. This list of materials is non limiting, and any suitable material or combination of materials may be used to construct the walls.

In some embodiments, the inner walls 19 may have readily cleanable finishes for gyms, med-spas, physical therapy facilities, hospitals, and personal home spaces. Optionally, inner walls 19 and outer walls 17 may be supported in a framing structure for case of construction, modularity, assembly, and/or installation.

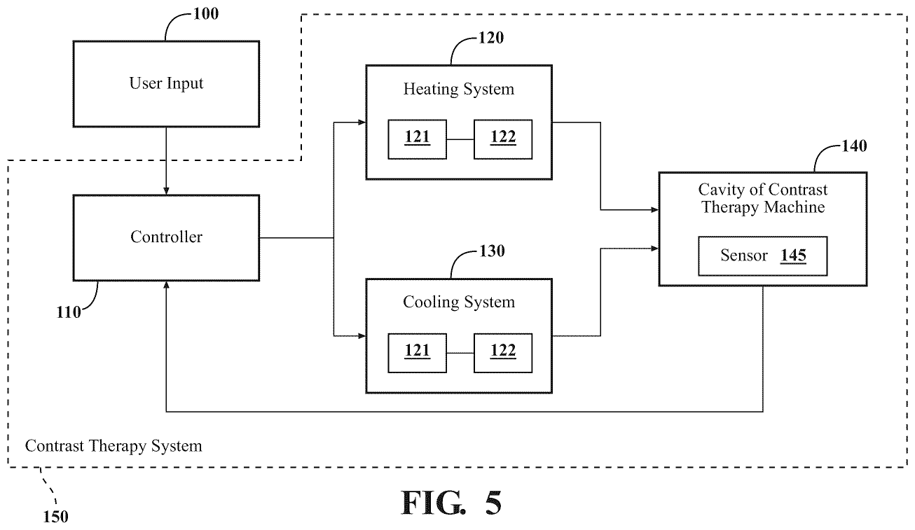

In FIG. 5, an example contrast therapy system 150 is shown. The contrast therapy system 150 accepts user input 100 for parameters as described above, whether delivered manually (such as by CP1 or CP2) or wirelessly through any of a number of communication protocols.

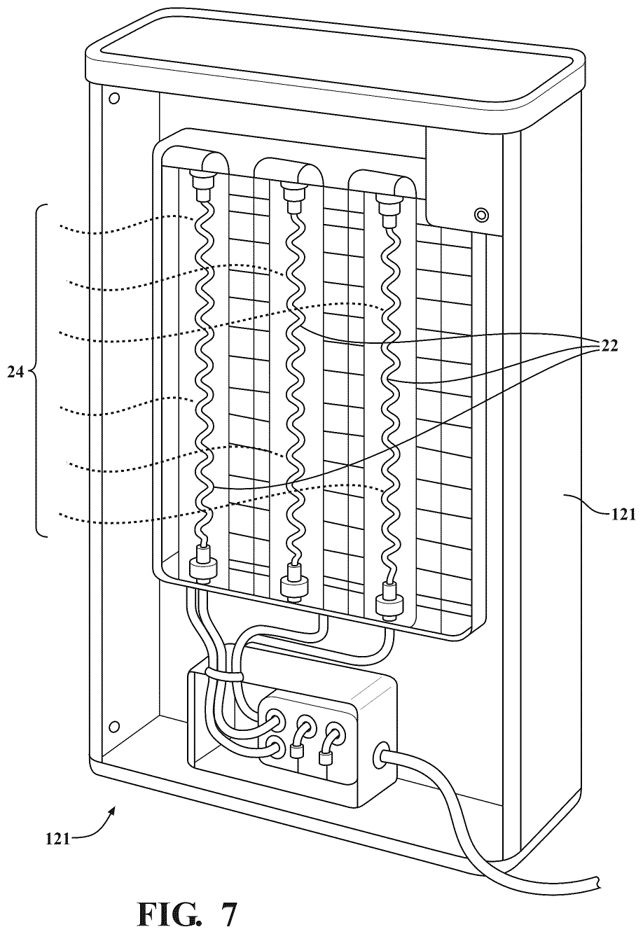

User input is received by controller 110, which may refer to one or more than one physical controllers or processors or microprocessors. Controller 110 includes or is in electrical communication with a regulator including or in communication with a thermostat and a timer. Controller 110 is also in electrical communication with, at least, components of a heating system 120, components of a cooling system 130, and one or more temperature sensors 145 in the cavity 140 of the contrast therapy machine 1. The controller 110 is configured to execute instructions stored on a non-transitory computer-readable medium, which causes the controller 110 to perform various functions and execute methods. For example, the controller 110 may process the received inputs to generate a control sequence, which may then define the timing, order, and selection of which components of the heating and cooling systems will be actuated, operated, and ceased. The controller 110 also receives inputs in the form of sensed temperature data from one or more temperature sensors 145 in the cavity 140. The controller 110 compares sensed temperature data to inputs and determines whether one or more component of a heating or cooling system needs to be actuated, operated, and ceased as conditions change in cavity 140. The controller 110 can then generate an output to modify or replace the initial control sequence.

Where a heating cycle is launched pursuant to the control sequence, depending on the inputs, one or more heaters 121 in heating system 120 may be actuated, which may include one or more of a heater 9 as shown in FIG. 2 or in more detail as heater 121 (FIG. 7), and optionally one or more of booster heaters 122, (FIG. 8). Depending on which types of heaters 121 are used or whether any booster heaters 122 are used, one or more additional fans may be used to move heated air. In some cases, heated air may move through channels or ducts in walls 5 to reach the cavity 140 of the contrast therapy machine 1. One or more sensors 145 in cavity 140 sense the temperature (and optionally other temperature data) in the cavity 140 and communicate sensed temperature data to the controller 110 for processing. When the peak temperature is reached as determined by the controller 110, then the controller 110 may directly or indirectly cause the turning off one or more of the heaters 121 or the booster heaters 122, only to cause them to be turned on again as may be useful to maintain the desired peak temperature for the selected duration of the heating cycle within a tolerance range. Because of the feedback loop, the peak temperature, as maintained, can be expected to have variation. Timing of heating cycles can be programmed to launch (begin a specified duration) from the moment a heater 121 is initiated in applications where precise quantities of time has greater importance than particular temperatures being reached. Such example applications may include, for example, commercial gyms or home installations. Timing of heating cycles can also be programmed to launch when the sensed temperature in the cavity 140 is within 5 degrees or fewer degrees of a selected temperature. This is for applications where a certain quantity of time within a particular temperature range is of greater importance than the total time spent in the cavity 140. Such example applications may include, for example, hospitals or PT/OT centers. Timing programming may be performed by the manufacturer or software provider or user.

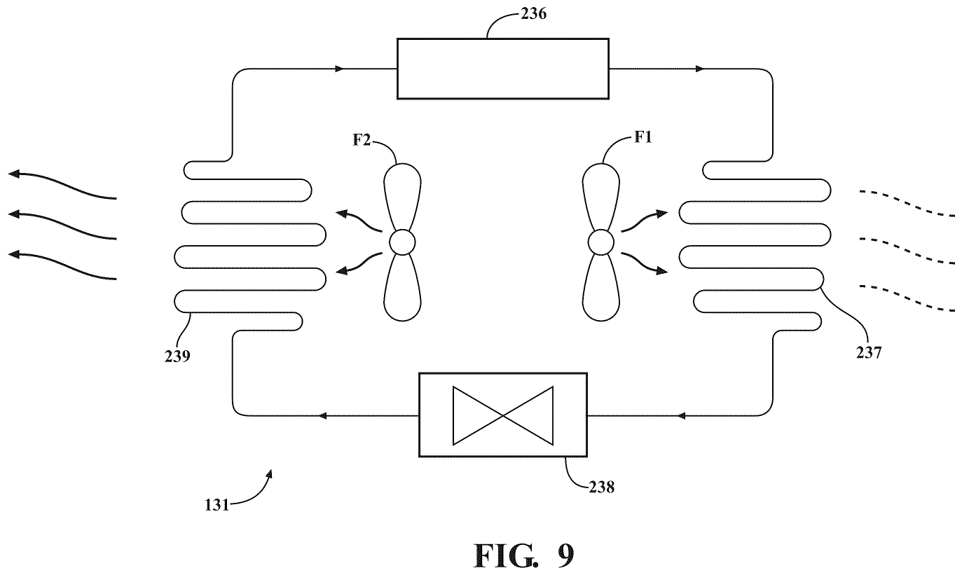

In some embodiments of heating system 120, peak temperatures may be set to 220 degrees Fahrenheit and reached by heaters 121 and booster heaters 122 within tolerances of +/−5 degrees F. or +/−3 degrees F. In some embodiments, peak temperatures may be set to 200 degrees F., 190 degrees F., 180 degrees F., 170 degrees F., and 160 degrees, F, 150 degrees F., 140 degrees F., and 120 degrees F. Other upper limits may be set. Once the heating system 120 completes a cooling cycle, one or more circulating and/or exhaust fans may be used to remove warmed air from the interior of the cavity 13 of contrast therapy machine 1, cooling the interior of the cavity 13 of contrast therapy machine 1.

Where a cooling cycle is launched pursuant to the control sequence, depending on the inputs, the controller causes, directly or indirectly, actuation of coolers 131 in cooling system 130, which may include one or more air conditioning units (an example of which is drawn in FIG. 9), and optionally one or more fans 132 to move heated air to one or more vents. Depending on which types of coolers 131 and fans 132 are used, one or more additional fans may be used to move cooled air through channels or ducts in walls 5 through bores to reach the cavity 140 of the contrast therapy machine. One or more sensors 145 in cavity 140 sense the temperature (and optionally other temperature data) in the cavity 140 and communicates sensed temperature data to the controller 110 for processing. When the nadir temperature is reached as determined by the controller 110, then the controller 110 may directly or indirectly cause the turning off one or more of the coolers 131 or fans 132, causing them to turn them on again only as may be useful to maintain the desired nadir temperature for the selected duration of the cooling cycle. Because of the feedback loop, the nadir temperature, as maintained, can be expected to have variation within tolerances. As with the heating cycles, timing for cooling cycles can be programmed to launch either upon initiation of a cooling cycle or upon reaching of nadir temperature within a particular range. Timing programming may be performed by the manufacturer or software provider or user.

In some embodiments of cooling system 130, coolers 131 can cool to and maintain set nadir temperatures as low as in the 20s, such as 22 or 28 degrees F. within tolerances of +/−5 degrees or of +/−3 degrees. In some embodiments, nadir temperatures may be set to 32.1 degrees Fahrenheit and reached by cooler 131 within tolerances. In some embodiments, nadir temperatures may be set to 60 degrees F., 55 degrees F., 50 degrees F., 45 degrees F., and 40 degrees, F, 35 degrees F., 30 degrees F., and 25 degrees F. Other lower limits are contemplated. Once the cooler completes a cooling cycle, one or more circulating and/or exhaust fans may be used to remove the cold air from the interior of the cavity 13 of contrast therapy machine 1, warming the interior of the cavity 13 of contrast therapy machine 1.

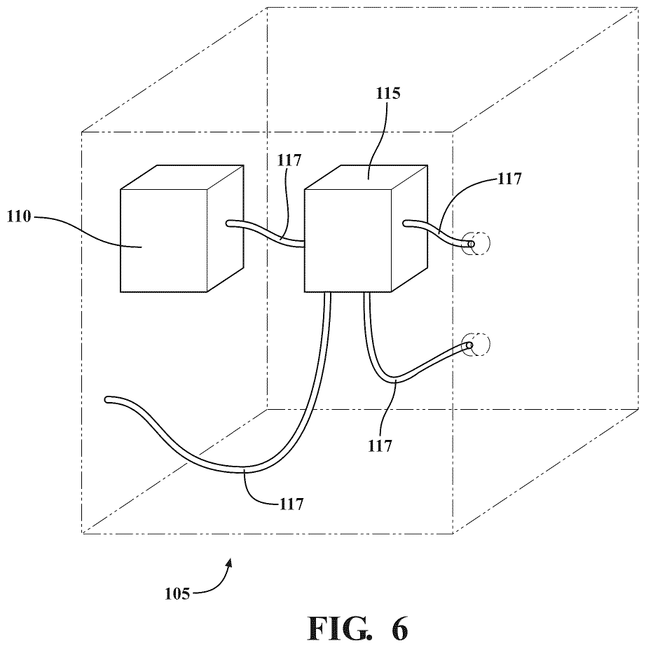

Referring to FIG. 6, an exemplary control box 105 is shown. Control box 105 may optionally house controller 110, which is in electrical communication with power source 115. Power source 115 may include any of a number power sources suitable for powering a contrast therapy machine 1. In some embodiments power source 115 is a direct or indirect connection to a 120V or 240V electrical outlet. Wires 117 may emanate from power source 115 to provide or cease providing power to components of the heating system 120 and the cooling system 130 as instructed by output of controller 110. Control box 105 is not shown on contrast therapy machine 1, but it is contemplated to reside in a recess in a wall 5 opposite door 2 or other inconspicuous location that is reasonably accessible for maintenance and repair.

Referring to FIG. 7, an exemplary heater 121 is illustrated. In this non-limiting example the heater is an IR heater where filaments 22 radiate heat as shown by heat waves 24. IR heaters tend to be low maintenance and run quietly. Other suitable heaters 121 with similar traits include, without limitation and only as by way of example, radiant panel heaters, oil-filled radiators, hydronic baseboard heaters, fanless ceramic heaters, underfloor radiant heating systems, and micathermic panel heaters.

Referring to FIG. 8, a booster heater 122 may be desired to reduce the time between nadir temperature or ambient air temperature and desired peak temperature. In such cases, one or more booster heaters 122 may be actuated based upon output from controller 110. As shown, ambient air 28 is pulled into booster heater 122 by fan 27 and forced over heating element 25 such as heat coils or filament 25. Booster heaters 122 may be noisier than heaters 121, and their use may optimized for the early stages of a heating cycle from an initiation of a contrast therapy program session or for the early stages of a heating cycle after transitioning from a cooling cycle. The booster heater 122 can also help bring the inside temperature to a higher heat range when used in conjunction with infrared wave heat panels, including as temperatures as high as 230 degrees Fahrenheit. The booster heater 122 may include its own thermostat 26, fan motor(s), and other components, some or all of which may be in electrical communication with a controller 110 and a power source 115. Booster heaters 122 may include forced-air electric heaters, ceramic heaters with fans, PTC (Positive Temperature Coefficient) fan heaters, blower-equipped convection heaters, propane or gas-fired space heaters with blowers, fan-assisted baseboard heaters, electric strip heat, or the like.

Referring to FIG. 9, a schematic of an exemplary cooler 131 is illustrated. Compressor 236 draws in refrigerant gas and compresses the gas. This compression increases both the pressure and temperature of the gas. The resulting hotter and high-pressure gas flows into condenser 237, which is typically located outside the cavity 13 of contrast therapy machine 1. A fan F1 blows air across the condenser 237, helping to dissipate heat from the refrigerant. As the refrigerant cools, it condenses into a high-pressure liquid. This liquid then passes through expansion valve 238, a restriction that reduces the refrigerant's pressure. The pressure drop causes some of the refrigerant to flash evaporate, lowering its temperature.

The cooled, low-pressure refrigerant enters evaporator 239. A second fan F2 pulls warm air from the cavity across the evaporator. As the refrigerant travels through the evaporator, it absorbs heat from the air and fully evaporates into a gas. During this process, moisture in the air may condense on evaporator coils, dehumidifying the air. The resulting cooled and dehumidified air is blown back into cavity 13, optionally through ducts or channels in walls 5. Condensation may be collected and directed outside the contrast therapy machine 1. The warm refrigerant gas then returns to compressor 236, and the cycle repeats for the duration of the programmed cooling cycle.

As noted supra, in some embodiments, cooler 131 is a self-contained air conditioning unit, such as a wall unit or mini-split system. Other suitable cooling mechanisms may include a Peltier thermoelectric cooler, a vapor compression refrigeration unit, an evaporative cooler, a cold air blower or cryo fan, cold plates, cold wall panels, or combinations of these or other known cooling devices. Cooler 131 may be in electrical communication with a controller and can be actuated, operated, or deactivated based on input and/or sensed temperature data processed by the controller.

Referring to FIG. 10, an exemplary seat base 307 and seat 308 is shown for use in contrast therapy machine 1. As depicted, seat base 307 and seat 308 are electronically vertically adjustable according to manual input in seat control box 310. As depicted, control box 310 has up and down buttons, and is in electrical communication with electric motor 353 that is engaged directly or indirectly with threaded seat base 307 to raise and lower seat 308 depending on input. Controls in control box 310 may optionally be in electrical communication with controller 110 and at least one power source 115.

Referring to FIG. 11, an exemplary seat base 407 and seat 408 is shown for use in contrast therapy machine 1. As depicted, seat base 407 and seat 408 are manually pneumatically vertically adjustable by use of seat handle 404. In this example, seat base 407 includes inner 42 and outer 41 chambers, piston 43, piston shaft 44, outer casing 47, mounting top 48, mounting base 50, and valve 45. Other components are omitted for simplicity. As the handle 404 is pulled, pressure is released and air or liquid passes through the valve 45, adjusting the height of seat 408. As the lever 404 is released, pressure is increased inside the lower portion of the inner chamber 42 underneath the piston 43 holding the height in place.

As noted supra, seats may come in a variety of sizes, shapes, with optional back supports any number of adjustable parts such as inflatable lumbar supports to accommodate a variety of users of different sizes, shapes, and physical conditions and abilities. In some embodiments, the seat is readily removable from contrast therapy machine 1 to accommodate wheel chairs.

### Contrast Therapy Programming

Users of contrast therapy machine can include home users, patrons of fitness clubs, and/or patients and therapists at physical and occupational therapy centers, including those internal to hospitals. The user interfaces for contrast therapy programming on CP1, CP2, a remote control, or a downloaded application on a phone, tablet, laptop or other computing device may be optimized for ease of use and set to include lower and upper limits based on a particular installation of heaters and coolers and fans.

Users may generally input preferences to generate a contrast therapy program session. Any of a number of inputs are possible, including but not limited to the below.

A user can select a number of heating cycles. As a non-limiting example, 0, 1, 2, or 3 heating cycles may be selected. Peak temperatures, within tolerances, can be selected. Where timing is set to commence at the reaching of a peak temperature, for example in a medical environment, the contrast therapy machine 1 may work like this. A user may select 2 heating cycles of 10 minutes each with a peak temperature of 200 degrees Fahrenheit. The 2 cycles can set for different durations and different peak temperatures. Using the 2 cycles at 10 minutes example, as a practical matter, it will take time to heat a contrast therapy machine 1 from an ambient temperature. So, from room temperature to +/−5 degrees of 200 degrees, it might take 5 or more minutes with IR heating panels and a booster heater to arrive at the peak temperature range (200 degrees Fahrenheit+/−5 degrees) to be maintained. If starting from a cooling cycle, it will take more time to arrive at the input peak temperature range. It is arrival at the peak temperature range when the timer begins the 10 minute cycle, and the booster heater goes off except as needed to maintain the temperature in the cavity 13. Thus, selecting two 10 minute heating cycles takes more than 20 minutes. This will not be the case where an installed contrast therapy machine 1 defaults to timing commencing at the beginning of actuation of a heating cycle. In such a case, two 10 minute heating cycles with take a total of 20 minutes, but some of that time early in the cycle, the temperature in cavity 13 will be lower than the selected peak temperature.

Similarly, a user can select a number of cooling cycles. As a non-limiting example, 0, 1, 2, or 3 cooling cycles may be selected. Nadir temperatures, within tolerances, can be selected. For example, a user may select 2 heating cycles of 10 minutes each with a nadir temperature of 35 degrees Fahrenheit. As a practical matter, where the timing is set to commence at reaching of the nadir temperature, it will take time to cool a contrast therapy machine 1 from an ambient temperature. So, from room temperature to +/−5 degrees of 35 degrees, it might take 5 or more minutes with an air conditioner to arrive at the nadir temperature range (35 degrees Fahrenheit+/−5 degrees) to be maintained. If starting from a heating cycle, it will take more time to arrive at the input nadir temperature range. It is arrival at the nadir temperature range when the timer begins the 10 minute cycle. Thus, selecting two 10 minute cooling cycles takes more than 20 minutes. This will not be the case where an installed contrast therapy machine 1 defaults to timing commencing at the beginning of actuation of a cooling cycle. In such a case, two 10 minute cooling cycles with take a total of 20 minutes, but some of that time early in the cycle, the temperature in cavity 13 will be higher than the selected nadir temperature.

A user can also select alert preferences. Preferences relate to type of alerts. Auditory alerts may be used, such as voice alerts or certain selected tones. Haptic alerts may be used, such as vibration of, for example, a seat in electrical communication with controller 110. Visual alerts may be selected. These may include warning lights or flashing light indicators. Visual alerts may also include wirelessly sent text messages. A user can select for which events alerts are sent. Events may be the beginning or end of a contrast therapy program session, the beginning or end of a heating cycle, the beginning or end of a cooling cycle, reaching of a peak temperature, reaching of a nadir temperature, or preliminary warnings of any of the foregoing.

A user can also select a sanitizing cycle before or after entry into contrast therapy machine in contrast therapy machines equipped with a sanitizer.

### Selected Modifications of Depicted Embodiments

Other embodiments are contemplated. For example, there are contrast therapy machines contemplated that do not include infrared heat panels. In such embodiments, hot and cold air may be pumped into vessels which, on demand, release air into the cavity quickly, which may have the impact of raising or lowering the inside temperature quickly. In embodiments without infrared heat panels, the remainder of the schematic of contrast therapy system remains generally the same.

Other components such as temperature sensors, regulators, alerts, alarms, and other safety measures are contemplated for inclusion. In some embodiments, a control for switching between a heating cycle and a cooling cycle can be pre-programmed or manually launched. In some embodiments, a regulator may be used to maintain temperatures within the contrast therapy machine within selected temperature ranges for selected durations. In some embodiments, the therapeutic time and temperature ranges are those that are therapeutically effective for whatever malady is being treated with the contrast therapy machine.

Reference throughout the specification to “embodiments” or “examples,” means that a particular system or component described in an example/embodiment is included in at least one embodiment. Such references are not necessarily all referring to the same embodiment. Furthermore, the features, structures, or characteristics may be combined in any suitable manner. Thus, the features, structures, or functions described with one example may be combined, in whole or in part, with the features, structures, and functions of one or more other examples if such combination is not illogical or non-functional.

While processes, systems, and methods may be described in connection with one or more steps in a particular sequence, such methods may be practiced with the steps in a different order, with certain steps performed simultaneously, with additional steps, and/or with certain described steps omitted.

References to a single element are not necessarily so limited and may include one or more of such element. Further, all numbers expressing dimensions, ratios and the like, used in the specification and claims, are to be understood to encompass tolerances and other deviations as represented by the term “about” or “substantially.” Any directional references (e.g., upper, lower, top, bottom) are only used to aid the reader's understanding of the present disclosure, and do not create limitations, particularly as to the position, orientation, or use of examples. Joinder references (e.g., attached, connected, etc.) may include intermediate members between a connection of elements, relative movement between elements, direct connections, indirect connections, fixed connections, movable connections, indirect contact, and/or direct contact. As such, joinder references do not necessarily imply that two components are directly connected.

The controller as described herein may include a conventional processing apparatus, which may be capable of executing preprogrammed instructions stored in an associated memory, all performing in accordance with the functionality described herein. The controller may include a memory on which computer-executable instructions may be stored, where the instructions may define operations and may be executable by one or more devices. Computer-executable instructions may be compiled or interpreted from computer programs created using a variety of programming languages and/or technologies.

The controller may be configured to perform various functions, including those described in greater detail herein, with appropriate programming instructions and/or code embodied in software, hardware, and/or other medium. The controller may include a wireless interface module that is configured to provide wireless communication to external devices. The wireless interface module may support wireless communication standards such as BLUETOOTH and/or wireless networking (Wi-Fi) as defined by Institute of Electrical and Electronics Engineers (IEEE) 802 family of standards (e.g., IEEE 802.11). The wireless interface module may be configured to transfer data between the controller and a remote device such as phone, tablet and/or computer. The controller may be programmed to implement a communications protocol that is compatible with the supported wireless communication standards.

To the extent that methods are embodied in software, the software can be stored in an associated memory and can also constitute means for performing such methods. Such a system or processor may further be of the type having ROM, RAM, and/or a combination of non-volatile and volatile memory so that any software may be stored and yet allow storage and processing of dynamically produced data and/or signals.

A memory may include, in general, any computer-readable medium (also referred to as a processor-readable medium) that may include any non-transitory (e.g., tangible) medium that provides instructions that may be read by a computer (e.g., by controller 110). Such a medium may take many forms, including, but not limited to, non-volatile media and volatile media. Non-volatile media may include, for example, optical or magnetic disks and other persistent memory. Volatile media may include, for example, dynamic random access memory (DRAM), which typically constitutes a main memory. Such instructions may be transmitted by one or more transmission media, including radio waves, metal wire, fiber optics, and the like, including the wires that comprise a system bus coupled to a processor of a computer. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read.

While a human body is depicted in the drawings and in the description, it is only one non-limiting example of the type of body that might benefit from the device as described. It is contemplated that any animal, such as a horse, dog, cat, cow or any other animal, may benefit from the device as described and the therapy that can be provided using the device.

1. A contrast therapy machine, comprising:
at least one wall, at least partially thermally insulated, supporting a door that surround a cavity that is sized and shaped to accommodate at least one body;
a top surface at least a portion of which covers the cavity in a closed position;
a temperature sensor within the cavity;
a heater for use in a heating cycle;
a cooler for use in a cooling cycle;
a controller adapted to receive input relating to one or more of number of heating cycles, duration of heating cycles, temperature peaks of heating cycles, number of cooling cycles, duration of cooling cycles, temperature nadir of cooling cycles; and
the controller being in electrical communication with at least one heater and at least one cooler, the controller further being in electrical communication with the temperature sensor;
the controller being adapted to, based at least upon the input and the sensed temperature data, process the input and generate output to cause one or more heaters or coolers to actuate, operate, or cease operation.

2. The contrast therapy machine of claim 1 further comprising a booster heater in electrical communication with the controller.

3. The contrast therapy machine of claim 1 wherein the cavity further includes a seat.

4. The contrast therapy machine of claim 2 wherein the top surface has an opening therethrough, and the seat is vertically adjustable.

5. The contrast therapy machine of claim 1 further comprising an interface for receiving manual input or remote input relating to one or more of number of heating cycles, duration of heating cycles, temperature peaks of heating cycles, number of cooling cycles, duration of cooling cycles, temperature nadir of cooling cycles.

6. The contrast therapy machine of claim 5 wherein the interface for receiving manual input is a touch screen.

7. The contrast therapy machine of claim 1 further comprising a sanitizer.

8. The contrast therapy machine of claim 7 wherein the sanitizer is one or more of a UV-C light sanitizer, an ozone generator, a dry steam cleaner, an electro static sprayer, a HEPA air purifier, or an activated carbon air purifier.

9. The contrast therapy machine of claim 7 wherein the controller is in electrical communication with a sanitizer and wherein the controller is adapted to receive input relating to a sanitizing cycle, including duration of the sanitizing cycle.

10. A contrast therapy machine, comprising:
a base, at least partially insulated walls extending from the base, a door supported by at least one of the walls, where the walls and door define a cavity therebetween, and a cover portion over the cavity;
a seat extending from the base;
at least one temperature sensor within the cavity;
an input receiver for accepting instructions for programming one or more of a cooling cycle and a heating cycle;
a heating system configured to generate heat according to received input and sensed temperature data; and
a cooling system configured to vent warm air and to generate cooling according to received input and sensed temperature data.

11. The contrast therapy machine of claim 10, wherein the cover includes an opening sized and shaped to accommodate a human neck, and wherein the seat is vertically adjustable.

12. The contrast therapy machine of claim 10, wherein the input receiver is a controller configured to receive manual or remote input from one or more of a touch screen on or in the contrast therapy machine, a handheld remote control, or a downloaded application installed on one or more of a phone, a laptop, a desktop, or a tablet.

13. The contrast therapy machine of claim 10, wherein the received input includes data including one or more of number of heating cycles, peak temperature for a heating cycle, duration for a heating cycle, number of cooling cycles, nadir temperature for a cooling cycle, and duration for a cooling cycle.

14. The contrast therapy machine of claim 13, wherein the peak temperature can be up to 220 degrees Fahrenheit.

15. The contrast therapy machine of claim 13, wherein the nadir temperature can be down to 20 degrees Fahrenheit.

16. The contrast therapy machine of claim 10, wherein the heating system comprises an infrared heater in electrical communication with a booster heater.

17. The contrast therapy machine of claim 10, wherein the cooling system comprises an air conditioner.

18. A non-transitory computer-readable medium storing instructions that, when executed by a controller, cause the controller to perform a method comprising:
a. receiving input indicating at least one of a number of heating cycles, a peak temperature, and a duration of each heating cycle for a contrast therapy program session;
b. receiving input indicating at least one of a number of cooling cycles, a nadir temperature, and a duration of each cooling cycle for the contrast therapy program session;
c. processing the received input to generate a control sequence for the contrast therapy program session;
d. actuating one or more heaters or one or more coolers to operate according to the control sequence;
e. receiving feedback in the form of sensed temperature data from one or more temperature sensors;
f. comparing the sensed temperature data to received input to determine if one or more heaters or one or more coolers requires adjustment in operation or cessation of operation; and
g. generating output to cause adjustment of the control sequence or operation of the heater or cooler as needed according to the comparison.

19. The method of claim 18 further comprising receiving input indicating preferences for alerts associated with the contrast therapy program session, and generating alerts based on the alert preferences.

20. The method of claim 19 wherein alerts may include one or more of auditory, including voice or tones, haptic, including vibration of an element in or on the contrast therapy machine, visual including light or flashing light indicators, or wireless text alerts for one or more of a beginning or end of the contrast therapy program session, a beginning or end of a heating cycle, a beginning or end of a cooling cycle, reaching of a peak temperature, reaching of a nadir temperature, preliminary warnings relating to any of the foregoing.
